(19) United States
(12) Patent Application Publication
(10) Pub. No.: US 2003/0114908 A1
(43) Pub. Date: Jun. 19, 2003

(54) Epicardial electrode lead, introducer for such a lead and set of instruments for electrode implantaion

(76) Inventor: Erhard Flach, Berlin (DE)

Correspondence Address: Patrick W. Rasche, Armstrong Teasdale LLP, Suite 2600, One Metropolitan Sq., St. Louis, MO, 63102, US

(21) Appl. No.: 10/323,047

(22) Filed: Dec. 18, 2002

(30) Foreign Application Priority Data
Dec. 19, 2001 (DE) 101 62 508.1

(51) Int. Cl. A61N 1/05

(52) U.S. Cl. 607/129

(57) ABSTRACT

Epicardial electrode lead (1) for minimally invasive implantation and anchoring to the epicardium, with an elongated supply lead (3) and an electrode head (5) distally attached thereto and substantially concentric therewith, wherein the electrode head comprises a fixation-hook section (7.1) that projects beyond its outer surface and is oriented substantially tangential to the electrode head, at an acute angle.

The invention relates to an epicardial electrode lead (for simplicity also termed “epicardial electrode”) according to the precharacterizing clause of Claim 1, as well as to accessories for implanting such a lead and fixing it to the epicardium in a minimally invasive operation.

Implantable electrode leads to be used in or at the heart have been developed in combination with implantable cardiac pacemakers and have long been known in a great variety of forms. By far the most significant of these are electrode leads to be disposed intracardially, having been guided into the heart directly by a transvenous route. For these electrodes diverse means of fixation to the inside wall of the heart or in the trabecular structure of the ventricle have been proposed and also put into practice.

Among these are various kinds of “screw-in” electrodes, which incorporate—preferably at the distal end—a corkscrew-shaped section for active fixation. In addition there are intracardial electrodes with barb or claw arrangements for atraumatic fixation to the trabeculae. Electrode leads are also known that are curved and/or branched in a special way and have a basic pre-formed configuration intended to ensure that the electrode makes close contact with the heart wall and hence will reliably transmit stimulation pulses from the pacemaker into the wall.

Furthermore, elaborate screw-in electrode structures have been described in which a corkscrew-shaped device is oriented perpendicular to the long direction of the electrode lead; as a consequence, it can penetrate the heart wall practically perpendicularly even though the associated lead is substantially parallel to the heart wall. It will be evident that such a construction is elaborate and relatively voluminous because of the need to change the direction of the driving force. This feature makes them unsuitable for practical employment in minimally invasive procedures.

Whereas intracardial electrodes are essentially the only type that are suitable for the purpuse of long term transmission of stimulus pulses from permanently implanted pacemakers, epicardial electrodes are used primarily for temporary stimulation of the heart during or after surgical interventions. They are also employed in the form of large-area “patch” electrodes in combination with implantable defibrillators. For the latter purpose, however, they have been widely used only in special constellations, because extremely complex open-heart surgery is required.

An increasingly significant application of epicardial electrodes is to transmit stimulation pulses to the left ventricle. Transvenous access to the left ventricle is complicated and difficult to achieve; the “classical” transvenous route runs through the superior vena cava into the right atrium, from the atrium into the coronary sinus and from the latter, by turning at approximately a right angle, into one of the venous coronary vessels on the left side of the body. To guide an intracardial electrode through the many bends and narrow places along the route demands extraordinary skill and great experience of the operator, and even then success is not guaranteed.

For this special site of application, namely the left ventricle, epicardial access in the course of a minimally invasive operation is fundamentally considerably easier to accomplish. In this case it is a matter of guiding the electrode into the space between the pericardium and the outer wall of the myocardium (the epicardium), without causing blood to enter the intrapericardial space. If such bleeding were to occur, it would produce a so-called tamponade—a large-area clot—which can very easily disturb the function and metabolism of the underlying myocardium. As soon as a suitable surgical technique can be designed, and sufficient experience has accumulated for this problem associated with puncture of the pericardium to be overcome, the application of an epicardial electrode to the left ventricle may well be preferable, in some circumstances, to intracardial implantation.

A known procedure for minimally invasive positioning of an epicardial electrode is to pass it through the skin and the underlying tissue of the patient and insert it through the pericardium so that the orientation of the electrode and/or its lead is automatically parallel to the myocardium, i.e. is “tangential”. In this position the screw-in electrodes known for intracardial employment are of just as little use as are other known mechanisms for the fixation of intracardial electrode leads.

Therefore epicardial electrodes with screws or hooks oriented perpendicular to their long direction have been proposed and even, to a certain extent, used in practice. Moreover, a method has become known in which an electrode with axially oriented distal fixation screw is screwed into the myocardium at an acute angle of ca. 30°, by means of suitable insertion instruments. It is easy to imagine that such an electrode would not do justice to the anatomical peculiarities of the myocardium, and that the arrangement would induce considerable long-term bending forces and punctate pressure peaks between electrode and myocardium, which would not be advantageous either for the electrode function or for the durability of the implant.

It is thus the objective of the invention to make available an improved epicardial electrode lead, which enables an anatomically correct and hence secure fixation. Furthermore, a set of implantation instruments suitable for this electrode lead is to be provided.

This objective is achieved with respect to the actual electrode by an epicardial electrode lead with the characteristics given in Claim 1, and with respect to suitable accessories by an introducer according to Claim 7 and a set of electrode implantation instruments according to Claim 13.

The invention includes the fundamental idea that at the distal end—the electrode head—of the lead a tangentially acting engagement element is provided, which can be screwed into the epicardium while the lead is oriented parallel to the epicardial surface. It further includes the idea of constructing this engagement element as a fixation-hook section.

In an embodiment that is preferred, from the present point of view, at least certain sections of the electrode head are enclosed in a helically coiled wire, which is spaced slightly apart from the electrode and the end of which is expanded and/or substantially stretched out to form the fixation-hook section. Thus the electrode head, in other words, bears a distal, axially oriented attachment screw of which at least the final “turn” has a greater lateral extent than the electrode head itself (a larger diameter, in the case of an electrode head that is circular in cross section). The free end of the screw projects radially beyond the wall of the electrode head and, with respect to the head, is tangentially oriented.

This construction is attractive in its simplicity and requires hardly any enlargement of the electrode head, which makes it particularly suitable for minimally invasive surgery. It is also significant that the simple structure keeps its production costs low.

For implantation the electrode is introduced into the space between pericardium and epicardium and moved into contact with the epicardium; then a suitably shaped guide wire is used to rotate the helical wire so that its free end (the fixation-hook section) penetrates or is hooked into the epicardium. By continuing the rotation, the subsequent “turns” of the wire are screwed into the epicardium, in which process they are consecutively bent elastically outward or expanded. The end result is that several sections of the screw wire are engaged with the epicardial tissue. Thus the electrode head is securely fixed to the epicardium.

In an embodiment alternative to that just described, the wire attached to the electrode head, the outer end of which forms the fixation-hook section, has a spiral configuration. An electrode with this construction is also economical and easy to implant.

The fixation method resembles that described above—with the difference that the spiral has only one section in engagement with the epicardium, and for fixation only a fraction of a complete rotation of the spiral or of the electrode head is needed. The stability of the anchoring, of course, cannot be as great as that obtainable with the above-mentioned, approximately cylindrical helical screw.

To facilitate fixation the fixation-hook section—i.e., in the specific case the above-mentioned helical or spiral wire—is made of a resilient, spring-like material and has a pointed end, or one provided with a cutting edge. This makes it possible for the operator to insert the wire into the epicardium by exerting very little force.

The introducer, proposed as an essential component of the set of implantation instruments mentioned above, advantageously ensures that as the fixation-hook section of the electrode is being rotated into the myocardium, it does not accidentally also penetrate the pericardium. That is, the electrode must be freely movable with respect to the pericardium. The proposed introducer is in principle equally suitable for both of the above-mentioned alternative embodiments of the fixation-hook section (as helical or spiral section).

The distal end of the introducer can be partially opened as far as its end face—which from the present viewpoint represents the preferred embodiment. In particular, the plastic body has a substantially cylindrical external shape, in particular the shape of a circular cylinder, and a distal end in substantially the shape of a section of a cylinder, in particular a half-cylinder.

The plastic body of the introducer is preferably stiffer in its distal end section than in the adjacent shaft section, because the latter must have a degree of flexibility to assist insertion (described in more detail below). Another feature that facilitates insertion is a beveled or rounded distal end face of the introducer, and the process of pushing the electrode lead through the introducer, once the latter has been put into place, is facilitated by a slippery coating in the lumen. Preferably the introducer is longitudinally curved in its distal end region such that it matches the contour of the heart, so as to enable the electrode lead to be apposed to the myocardium as “smoothly” as possible, and thus to make fixation both easy and secure.

To perform the fixation, i.e. to rotate the fixation-hook section so that it is screwed into the myocardium, as a component of the set of implantation instruments a special guide wire (mandrel) is provided, which can be brought into rotationally stable engagement with the fixation-hook section—or with the entire electrode head, insofar as the fixation-hook section is nonrotatably attached thereto. At its proximal end there is an actuating section to transfer torque to the fixation-hook section (or electrode head) and to determine its precise angular position. In an especially simple embodiment this is a screwdriver-type engagement section constructed according to one of the customary standards (for slotted-head, cross-head, hexagonal etc. screws).

Additional advantages and useful features of the invention will be evident from the subordinate claims as well as the following description of preferred exemplary embodiments with reference to the figures, wherein

FIG. 1 is a perspective drawing of the end of an epicardial electrode lead according to a first embodiment,

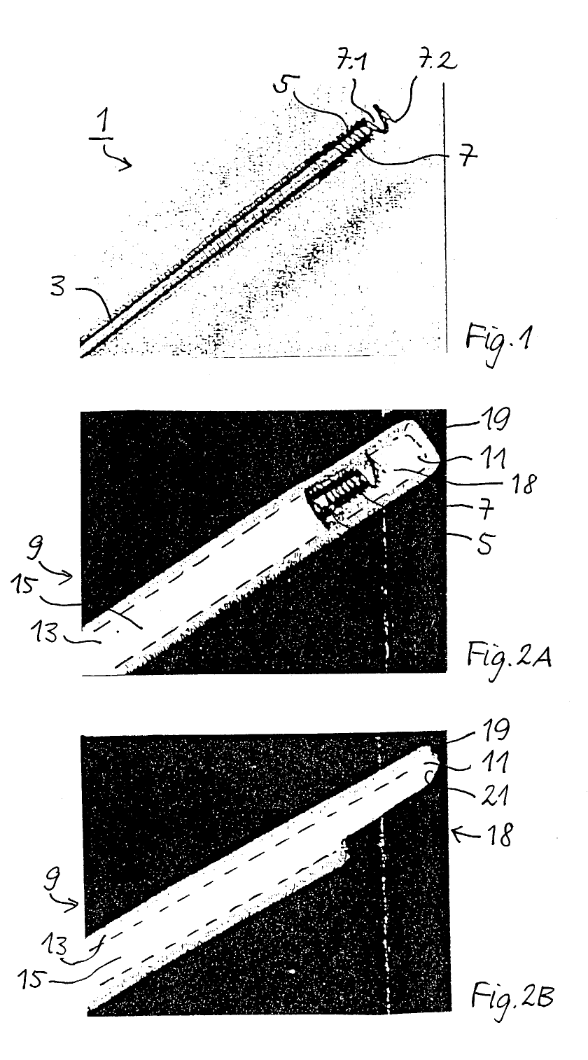

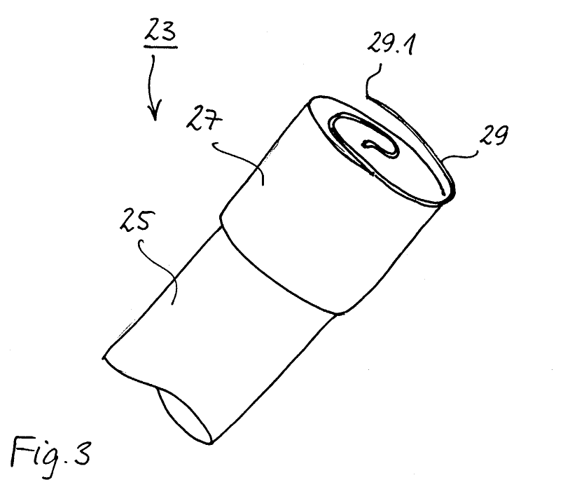

FIGS. 2A and 2B show two views of the distal end of an introducer to be used with the electrode lead according to FIG. 1, and

FIG. 3 is a sketch of the distal end of an epicardial electrode lead according to another embodiment.

FIG. 1 shows the distal end section of a substantially cylindrical electrode lead 1, which comprises a supply lead 3 and an electrode head 5. The electrode head 5 is surrounded over most of its length by a cylindrically coiled wire, the helical screw 7, the last turn 7.1 of which is somewhat extended in the longitudinal direction as well as expanded beyond the maximal diameter of the electrode head 5. The distal end 7.2 of the helical screw is sharpened.

In FIGS. 2A and 2B are two views of an introducer 9 made of plastic, with a hard or stiff tip 11 and a moderately hard or semi-stiff shaft 13. The introducer 9 is substantially cylindrical over its entire length; its lumen 15 is provided with a slippery coating and has a diameter matched to the (maximal) outside diameter of the electrode lead 1, i.e. the diameter of the last turn 7.1 of the helical screw 7. In FIG. 2A the introducer 9 is shown with the electrode lead 1 pushed into its lumen, so that the electrode head 5 with helical screw 7 is visible. That is, the electrode head is situated in the distal end region 17 of the introducer 9, which is shaped like a half cylinder, having an elongated opening 18 on one side that extends to its distal end face 19. There a beveled surface 21 is provided to facilitate insertion of the introducer during the minimally invasive operation described below.

For implantation of the electrode lead 1 according to FIG. 1 with the aid of the introducer 9 according to FIGS. 2A and 2B, the following method of access is proposed:

1. Local anesthesia below the sternum and puncture of the pericardium.

2. X-ray visualization of the heart by injection of contrast media into the pericardial sac.

3. Probing the pericardial sac with a Seldinger wire.

4. Introduction of a sheath by means of a dilator.

5. Introduction of a controllable ablation catheter to control the position of the peel-away sheath for temporary stimulation.

6. Visualization of the coronary arteries by an intravascular access route, to avoid injury to the vessels by possible active electrode fixations.

7. After the end of the guide catheter has been positioned, the controllable ablation catheter is exchanged for the permanently implantable electrode.

The sketch in FIG. 3 shows the principle of a modified electrode lead 23 with a supply lead 25 and an electrode head 27, which are similar to those of the electrode lead 1 according to FIG. 1. The substantial difference lies in the fact that in the present embodiment, instead of a helical screw coiled around the periphery of the electrode head, there is provided a spiral wire 29 attached to the electrode head at its distal end; the outer end of the spiral extends radially beyond the circumference of the electrode head 27 and is substantially tangentially oriented. The spiral wire 29, again, has a pointed end 29.1. For implantation substantially the same access method as outlined above can be employed.

The implementation of the invention is not restricted to the examples described above and aspects emphasized there, but is also possible in a large number of modifications that are within the competence of a person skilled in the art.

### List of Reference Numerals

1; 23 Electrode lead

3; 25 Supply lead

5; 27 Electrode head

7 Helical screw

7.1 Last turn

7.2; 29.1 Pointed end

9 Introducer

11 Tip

13 Shaft

15 Lumen

17 Distal end region

18 Side opening

19 Distal end face

21 Beveled surface

29 Spiral wire

1. Epicardial electrode lead (1; 23) for minimally invasive implantation and anchoring to the epicardium, with an elongated supply lead (3; 25) and an electrode head (5; 27) distally attached thereto and substantially concentric therewith, characterized in that the electrode head comprises a fixation-hook section (7.1; 29) that extends beyond its outer surface and is oriented substantially tangential to the electrode head, at an acute angle.

2. Epicardial electrode lead according to claim 1, characterized in that the electrode head (5) is surrounded over at least part of its length by a helical wire (7) spaced slightly apart from the electrode head and having an end (7.1) that is expanded and/or substantially extended and that forms the fixation-hook section.

3. Epicardial electrode lead according to claim 1, characterized in that to the electrode head (27) a spiral wire (29) is attached, the outer end of which forms the fixation-hook section.

4. Epicardial electrode lead according to claim 1, characterized in that the electrode head (5; 27) has substantially the shape of a cylinder, sphere or truncated cone and the fixation-hook section (7.1; 29) runs substantially tangential to the surface of the electrode head.

5. Epicardial electrode lead according to claim 1, characterized in that the fixation-hook section (7.1; 29), in particular the entire helical wire or spiral wire, has a resilient, spring-like construction.

6. Epicardial electrode lead according to claim 1, characterized in that the fixation-hook section (7.1; 29) comprises a distal end with a sharp tip or cutting edge (7.2; 29.1).

7. Introducer (9) for minimally invasive implantation and anchoring of an epicardial electrode lead (1; 23) according to claim 1, with an elongated plastic body (11, 13) matched to the shape and dimensions of the epicardial electrode lead and having a distal end that is open at the side (18) such that the fixation-hook section (7.1; 29) of the epicardial electrode lead can, by rotating the lead within the introducer, be brought into engagement with a section of epicardial tissue adjacent to the opening.

8. Introducer according to claim 7, characterized in that the plastic body (11, 13) has an external surface in substantially the shape of a cylinder, in particular a circular cylinder, and a distal end (17) in substantially the shape of a section of a cylinder, in particular a half-cylinder.

9. Introducer according to claim 7, characterized by a stiff distal end section (11), immediately proximal to which is a semi-stiff shaft section (13).

10. Introducer according to claim 7, characterized by a beveled (21) or rounded distal end surface (19).

11. Introducer according to claim 7, characterized by a slippery coating of a lumen provided to contain the epicardial electrode lead.

12. Introducer according to claim 7, characterized by a longitudinal curvature of the distal end section that is matched to the surface contour of the heart.

13. Set of electrode implantation instruments with an epicardial electrode lead (1; 23) according to claim 1 and an introducer (9) according to claim 7.

14. Set of electrode implantation instruments according to claim 13, with a guide wire that can be nonrotatably coupled to the electrode head of the epicardial electrode lead, at the proximal end of which is provided an actuation section for transmitting torque to the electrode head and for precisely determining its angular position.

15. Set of electrode implantation instruments according to claim 14, characterized in that the actuator section has a configuration such as is standard for the engagement end of a screwdriver.
